(19) United States
(12) Patent Application Publication
(10) Pub. No.: US 2023/0089469 A1
(43) Pub. Date: Mar. 23, 2023

(54) SYSTEM AND METHOD FOR DECREASING EFFECTS OF LACK OF MUSCLE USE

(71) Applicant: MULTI RADIANCE MEDICAL, Solon, OH (US)

(72) Inventors: Douglas JOHNSON, Brownstown, MI (US); Max KANARSKY, Solon, OH (US); Ernesto Cesar Pinto LEAL JUNIOR, Sao Paulo (BR)

(21) Appl. No.: 17/799,886

(22) PCT Filed: Feb. 12, 2021
(86) PCT No.: PCT/US2021/017952

§ 371 Date: Aug. 15, 2022

Related U.S. Application Data
(60) Provisional application No. 62/975,849, filed on Feb. 13, 2020.

(51) Int. Cl. A61N 2/00; A61N 5/06; A61N 2/06

(52) U.S. Cl. CPC A61N 2/002; A61N 2/004; A61N 2/06; A61N 5/0613; A61N 2005/066; A61N 2005/0652; A61N 2005/0663

(57) ABSTRACT

During a period of lack of use, a person risks muscle atrophy and/or otherwise losing muscle strength/functionality. During such a period of lack of user, a photoceutical can be applied to the person’s muscle(s) through a photoceutical medical device to decrease the occurrence of these risks. The photoceutical is specially configured to reduce these risks and includes at least one of a pulsed light signal, a continuous light signal, and a super-pulsed light signal.

### CROSS-REFERENCE TO RELATED APPLICATIONS

This application is a U.S. National Stage application under 35 USC 371, of PCT Application Serial No. PCT/US21/17952, filed Feb. 12, 2021, which claims the benefit of U.S. Provisional Application No. 62/975,849, filed Feb. 13, 2020, each of which are hereby incorporated by reference in their entirety for all purposes.

### TECHNICAL FIELD

The present disclosure relates generally to the effects of lack of muscle use and, more specifically, to systems and methods for decreasing the effects caused by lack of muscle use, including loss of strength, loss of functional capacity, loss of performance, reducing the effects of muscle atrophy, preserving muscle morphology, and/or preventing muscle atrophy with a photoceutical applied through a photoceutical medical device during a period of the lack of muscle use.

### BACKGROUND

Regular exercise has many positive benefits to a person, including increasing strength and improving functional capacity and performance. In the event of illness, injury, or other factors, the person may stop exercising or stop moving all together. Such a stoppage of exercise and other instances of lack of use can cause a rapid loss of muscle strength, functional capacity, and/or performance and may even lead to muscle atrophy. Even when the person has not engaged in regular exercise, a long period of inactivity (e.g., illness, injury, hospitalization, or the like) can also lead to muscle atrophy and other negative consequences.

### SUMMARY

The present disclosure relates to decreasing the effects caused by lack of muscle use, including loss of strength, loss of functional capacity, loss of performance, reducing the effects of muscle atrophy, preserving muscle morphology, and/or preventing muscle atrophy with a photoceutical applied through a photoceutical medical device during a period of the lack of muscle use. The loss of strength, loss of functional capacity, loss of performance, the effects of muscle atrophy due to lack of use can be decreased, muscle morphology can be preserved, and/or muscle atrophy can be prevented by application of a photoceutical through a photoceutical medical device during a period of the lack of use. The photoceutical medical device can include (1) one or more infrared super pulsed laser light sources, (2) one or more infrared light emitting diodes (IREDs), (3) one or more light emitting diodes (LEDs), and, in some instances, (4) one or more magnets to deliver the photoceutical comprising one or more of a super-pulsed light, an IR light, a red light, and, in some instances, a static magnetic signal.

In one aspect, the present disclosure can include a method for decreasing the loss of strength, functional capacity, and/or performance, decreasing and/or preventing muscle atrophy due to lack of use, and/or preserving muscle morphology. A photoceutical medical device can be contacted to a spot on a patient’s skin over at least a portion of a muscle during a detraining period after a physical activity has been discontinued. A photoceutical can be applied through the photoceutical medical device to the muscle from a start time to an end time to preserve strength and morphology of the muscle gained from the physical activity and/or prevent atrophy of the muscle during the detraining period. The photoceutical includes at least one of a pulsed light signal, a continuous light signal, and a super-pulsed light signal.

In another aspect, the present disclosure can include a photoceutical medical device that can act as the photoceutical delivery device. The device can include a circuit board comprising: a plurality of light sources to provide a light signal and at least two magnets to provide a magnetic signal. The plurality of light sources can include at least one super pulsed laser to provide super-pulsed light of a first wavelength, at least two non-coherent light sources to provide light of a second wavelength, at least two other non-coherent light sources to provide light of a third wavelength, such that the light signal includes at least one of the super-pulsed light of the first wavelength, the pulsed and/or continuous light of the second wavelength, and the pulsed and/or continuous light of the third wavelength. The light signal and the magnetic signal are delivered as a photoceutical to a spot on skin of a patient over at least a portion a muscle during a detraining period after a physical activity is discontinued from a start time to an end time to preserve strength of the muscle and morphology of the muscle gained from the physical activity and/or prevent atrophy of the muscle during the detraining period. The device can also include a power source (which may be at least partially on or in communication with the circuit board).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing and other features of the present disclosure will become apparent to those skilled in the art to which the present disclosure relates upon reading the following description with reference to the accompanying drawings, in which:

FIG. 1 is a diagram showing an example of a system including a photoceutical medical device that delivers a photoceutical to at least one muscle during a detraining period to decrease the loss of strength, functional capacity, performance, and/or muscle atrophy due to lack of use in accordance with an aspect of the present disclosure;

FIG. 2 is a diagram showing an example interior of the photoceutical medical device of FIG. 1;

FIG. 3 is an example photograph of a MR5Active Pro LaserShower device;

FIG. 4 is an example photograph with components enlarged of the light delivery surface of the MR5Active Pro LaserShower device;

FIGS. 5 and 6 are example diagrams of different photoceutical medical devices;

FIGS. 7 and 8 are process flow diagrams of an example method for delivering a photoceutical to a patient during a detraining period to decrease the loss of strength, functional capacity, performance, and/or muscle atrophy due to lack of use in accordance with another aspect of the present disclosure;

FIGS. 9 and 10 are graphs showing results of a maximum voluntary contraction (MVC) test;

FIGS. 11 and 12 are graphs showing results of a one-repetition maximum (1RM) test for leg extension; and

FIGS. 13 and 14 are graphs showing results of a 1RM test for leg press.

### DETAILED DESCRIPTION

### 1 Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

As used herein, the terms “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “detraining” refers to the partial or complete loss of training-induced adaptations (e.g., physical conditioning) due to a training stimulus that is insufficient or removed entirely. While typically used to refer to a period when exercise is stopped (e.g., a period of lack of training for a marathon because of a minor injury), detraining can also refer to general periods of inactivity (e.g., a period of lack of activity due to illness, injury, hospitalization, ventilation, or the like). For example, an individual who discontinues physical activities due to illness, injury or other factors may lose physical conditioning due to detraining.

As used herein, the term “photoceutical” refers to a light signal (or a light signal and magnetic signal) used to change a function of at least a portion of a patient’s body (e.g., by photobiomodulation to induce a phototherapeutic response using a drug-free, non-invasive treatment procedure). For example, the light signal of the photoceutical may include a combination of a plurality of light signals from a plurality of light sources, each of the plurality of light signals may have a different wavelength, frequency, or other property. The combination of the different properties (like wavelength, for example) can create a synergistic effect. As another example, the light signal can include one or more of the plurality of light signals from the plurality of light sources.

As used herein, the terms “photoceutical delivery device”, “photoceutical medical device”, “photoceutical device”, and the like, refer to a device configured to apply the photoceutical as a therapeutic agent. For example, the photoceutical delivery device can house the plurality of light sources (and, in some instances, one or more magnets) to deliver the photoceutical. The light sources can include one or more super pulsed lasers, one or more light emitting diodes, one or more infrared light emitting diodes, or the like. The one or more magnets can be permanent magnets, electromagnets, or the like.

As used herein, the term “super-pulsed laser” refers to a light source that produces a wavelength of light at a high peak power (e.g., as high as 50,000 mW) for a very brief duration (e.g., each pulse is for a billionth of a second), leading to a high concentration of photons driven deeply into target tissue without the risk of overheating. Even though the pulse peaks at a high power level, there are no thermal effects in the tissue. The peak power is high compared to the average output power. By using a super pulsed laser, one is able to more effectively deliver higher densities of light energy into the tissue without associated deleterious thermal effects. Super-pulsing can allow for deeper penetration depths than a pulsed or continuous light signal.

As used herein, the term “muscle” refers to any soft tissue within the body composed of cells or fibers that contract to change a length and/or a shape of the soft tissue. The muscle can produce movement in the body. The muscle can be skeletal muscle, smooth muscle, and/or cardiac muscle.

As used herein, the term “effect” refers to a change that is a result or consequence of an action or other cause. The effect of lack of muscle use can be loss of strength, loss of morphology, loss of functional capacity, loss of performance, muscle atrophy, or the like. As an example, lack of muscle use due to a period of mechanical ventilation or any other lung malady (e.g., chronic obstructive pulmonary disease (COPD), acute respiratory distress syndrome (ARDS), etc.) can lead to loss of strength and/or muscle atrophy. As another example, lack of muscle use during a detraining period for an elite athlete can lead to loss of functional capacity, loss of morphology and/or loss of performance.

As used herein, the term “sufficient” refers to an amount adequate enough to satisfy a condition.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc. The patient can require mechanical ventilation. In some instances, the subject can be in a detraining period, in which the subject has stopped or minimized exercise due to illness, injury or other factors.

### II. Overview

During a period of lack of muscle use, a person can lose muscular strength, morphology, functional capacity, performance, or the like, and/or suffer from muscular atrophy due to the lack of use. For example, the period of lack of muscular use can be a detraining period, when the person suffers the partial or complete loss of training-induced adaptation (e.g., physical conditioning) due to the training stimulus being insufficient or removed entirely. It should be understood that while typically used to refer to a period when exercise is stopped (e.g., a period of lack of training for a marathon because of a minor injury), detraining can also refer to general periods of inactivity (e.g., a period of lack of activity of a person who is not an elite athlete, or even an athlete at all, due to hospitalization, mechanical ventilation, disease, injury, or the like). For example, an individual who discontinues physical activities due to illness, injury, or other factors may lose physical conditioning due to detraining. Described herein are systems and methods for decreasing loss of muscular strength, functional capacity, performance, morphology, or the like, and/or muscle atrophy due to lack of muscle use with a photoceutical applied through a photoceutical medical device during a period of the lack of use.

The photoceutical can include a light signal that can be a combination of one or more lights from one or more light sources, each having unique parameters (e.g., wavelength, frequency, or other property). In some instances, the photoceutical can also include a magnetic signal. For example, the light signal can include super-pulsed light of a first wavelength, light of a second wavelength, and light of a third wavelength (two of the wavelengths can be substantially matched and one wavelength can be different). The photoceutical can be delivered by a photoceutical medical device that includes at least one super pulsed laser to provide super-pulsed light of the first wavelength, at least two non-coherent light sources to provide light of the second wavelength, and at least two other non-coherent light sources to provide light of a third wavelength (and may, additionally, include one or more magnets to provide a magnetic signal).

The photoceutical can be delivered to a patient (who is in a detraining period after physical activity has been discontinued or who is otherwise unable to perform physical activity) by a photoceutical medical device. The photoceutical medical device can be contacted to a spot on a patient’s skin over at least a portion of a muscle to deliver the photoceutical. The photoceutical can be applied through the photoceutical medical device to the muscle from a start time to an end time to decrease the effects caused by lack of muscle use, including loss of strength, loss of functional capacity, loss of performance, effects of muscle atrophy, to preserve muscle morphology, and/or to prevent muscle atrophy.

### III Photobiomodulation Therapy (PBMT)

One or more doses of a photoceutical can be applied to a patient during a detraining period (e.g., a period of reduced/no exercise/activity) to decrease the loss of strength, loss of functional capacity, loss of performance, effects of muscle atrophy due to lack of use, preserve muscle morphology, and/or prevent muscle atrophy. The photoceutical is a drug-free and non-invasive photobiomodulation therapy (PBMT) that includes a light signal (and, in some cases, a magnetic signal) specifically configured to treat the patient during the detraining period. The photoceutical can be applied through the skin in a non-invasive manner to treat such patients by inducing a phototherapeutic response. In this case, the phototherapeutic response can camouflage (for a time) the fact that the patient has ceased exercise/activity, preventing the patient from losing strength, losing functional capacity, losing performance, preserving muscle morphology, and/or experiencing muscle atrophy due to lack of use.

The light signal of the photoceutical may be configured to include a combination of a plurality of light signals from a plurality of light sources, each of the plurality of light signals may have a different wavelength, frequency, or other property. The combination of the different properties (like wavelength, for example) can create a synergistic effect. For example, the light signal can include a combination of a super-pulsed light of a first wavelength (850 nm - 950 nm), light of a second wavelength (800 nm - 900 nm), and light of a third wavelength (580 nm - 800 nm). In some instances, the light signal also includes a magnetic signal (or magnetic field). A dose of the light signal can be applied for a time period from a first time to a second time, wherein the time period is between 30 seconds to 1 hour, to each of a number of unique sites over a muscle. The three different wavelengths can be selected to cover the entire spectrum of the therapeutic window of light for deeper penetration and enhanced absorption of light. The penetration to the target area is based on specific characteristics of the different light signals (e.g., wavelengths, powers, etc.) and, in some instances, the magnetic signal.

The light of PBMT has been shown to have a modulatory effect on muscle cells based on the principle that certain molecules in living systems absorb photons and trigger signalling pathways in response to light. When a photon of light is absorbed by a chromophore in a cell, an electron in the chromophore can become excited and jump from a low-energy orbit to a higher-energy orbit. This stored energy then can be used by the living system to perform various cellular tasks, such as cellular metabolism, microcirculation, promoting oxygen availability, and modulation of the inflammatory process, attributable to the acceleration of the electron transport chain and reestablishment of oxidative phosphorylation. While not wishing to be bound by theory, there is strong evidence to suggest that one of the basics of PBMT is the acceleration of electron transfer by electromagnetic radiation in the visible and near infrared region of the spectrum, via the modulation of cytochrome c-oxidase (“CCO”) activity in muscle cells. CCO is the primary photo acceptor of visible to near infrared light energy and is the enzyme responsible for catalysing oxygen consumption in cellular respiration and for the production of nitric oxide under hypoxic conditions. High-energy electrons are passed from electron carriers through a series of trans-membrane complexes (including CCO) to the final electron acceptor, generating a proton gradient that is used to produce adenosine triphosphate (ATP). The application of light directly results in ATP production and electron transport. In short, the application of PBMT can increase ATP production, down-regulate cellular respiration modulated by nitric oxide (NO), and promotes the metabolism of oxygen, while increasing the production of reactive oxygen species (ROS).

While not wishing to be bound by theory, it is believed that PBMT can cause a phototherapeutic response that can camouflage (for a time) the fact that the patient has ceased exercise/activity due to the PBMT modulating mitochondrial activity through interaction with cytochrome c oxidase (CCO), increasing the adenosine triphosphate (ATP) production, which in turn, accelerates cell metabolism.

### IV. Systems

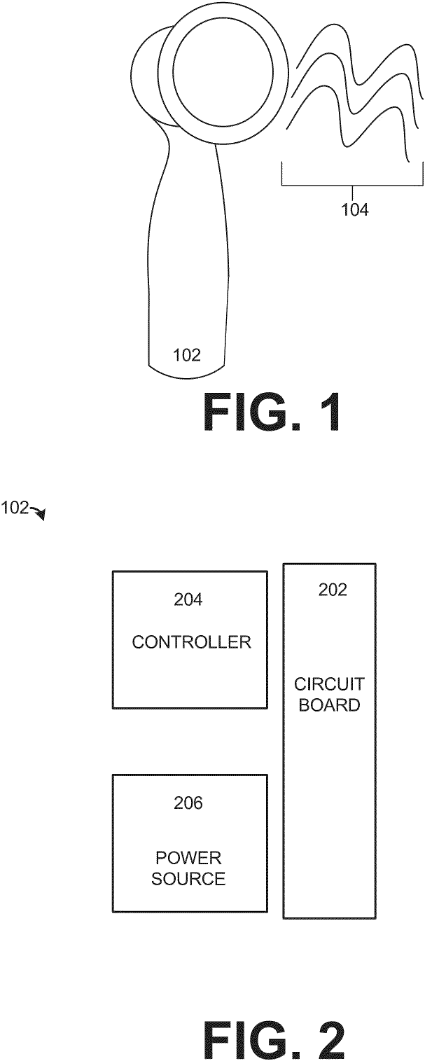

Described herein is a system that can be used to decrease the effects caused by lack of muscle use, including loss of strength, loss of functional capacity, loss of performance, effects of muscle atrophy, prevent muscle atrophy, and/or preserve muscle morphology with a photoceutical 104 applied through a photoceutical medical device 102 (shown in FIG. 1) during a period of the lack muscle of use. As shown in FIG. 1, the photoceutical medical device 102 can provide and deliver a photoceutical 104 that can be configured to decrease the effects caused by lack of muscle use during a detraining period or other period of lack of use (e.g., a period where a patient is unable to perform a physical activity due to an injury, a disease, like chronic obstructive pulmonary disease, a period of hospitalization, a period of mechanical ventilation, or the like). The photoceutical 104 can be delivered to one or more muscles identified as potentially subject to the effects caused by lack of muscle use based on specifics of the patient in a period of detraining or any other period of lack of use. For example, based on the patient, one or more leg muscles and/or one or more muscles used for respiration (e.g., chest muscles, neck muscles, the diaphragm, etc.) can be identified as targets. The photoceutical medical device 102 can be contacted to a spot on a patient’s skin over at least a portion of each selected muscle to deliver the photoceutical 104. The photoceutical 104 can be applied through the photoceutical medical device to the muscle at each spot from a start time to an end time to decrease the effects caused by lack of muscle use, including loss of strength, loss of functional capacity, loss of performance, effects of muscle atrophy, prevent muscle atrophy, and/or preserve muscle morphology.

The photoceutical 104 can include a light signal that can be a combination of one or more lights from one or more light sources, each having unique parameters (e.g., wavelength, frequency, or other property). For example, the light signal can include super-pulsed light of a first wavelength, light of a second wavelength, and light of a third wavelength (two of the wavelengths can be substantially matched and one wavelength can be different). In some instances, the photoceutical 104 can also include a magnetic signal.

The photoceutical 104 can be delivered to each target by the photoceutical medical device 102. The photoceutical medical device 102 can include at least one super pulsed laser to provide super-pulsed light of the first wavelength, at least two non-coherent light sources to provide light of the second wavelength, and at least two other non-coherent light sources to provide light of a third wavelength. For example, the first wavelength can be any wavelength between 850 nm and 950 nm, the second wavelength can be any wavelength between 800 nm and 900 nm, and the third wavelength can be any wavelength between 580 nm and 800 nm. The processor can choose a time sequence at which the three wavelengths of light are delivered. The three wavelengths can be chosen to manipulate the interaction between the wavelengths to achieve the desired penetration of light to the target. For example, the third wavelength can be absorbed by superficial tissue to clear the way for the second wavelength to penetrate deeper and eliminate cellular interference, which can allow the first wavelength to go deeper into the tissue.

The photoceutical medical device 102 can also include at least two magnetic sources (at least two magnets, e.g., that provide a static magnetic field from 5 mT to 1 T) to provide the magnetic signal (e.g., a magnetic field). In some instances, the photoceutical medical device 102 can include at least four super pulsed lasers to provide super-pulsed light of the first wavelength, at least eight non-coherent light sources to provide light of the second wavelength, and at least eight other non-coherent light sources to provide light of a third wavelength. In these instances, the photoceutical medical device 102 can also include at least eight magnetic sources to provide the magnetic field.

The photoceutical can include a light signal from one or more of the light sources and also may include the magnetic signal. The light signal can include at least one of the super-pulsed light of the first wavelength, the pulsed and/or continuous light of the second wavelength, and the pulsed and/or continuous light of the third wavelength. In some instances, the light signal can include the super-pulsed light signal and another signal from the at least two non-coherent light sources or the other at least two non-coherent light sources. The wavelengths and the powers can be coordinated to enhance effects of the photoceutical.

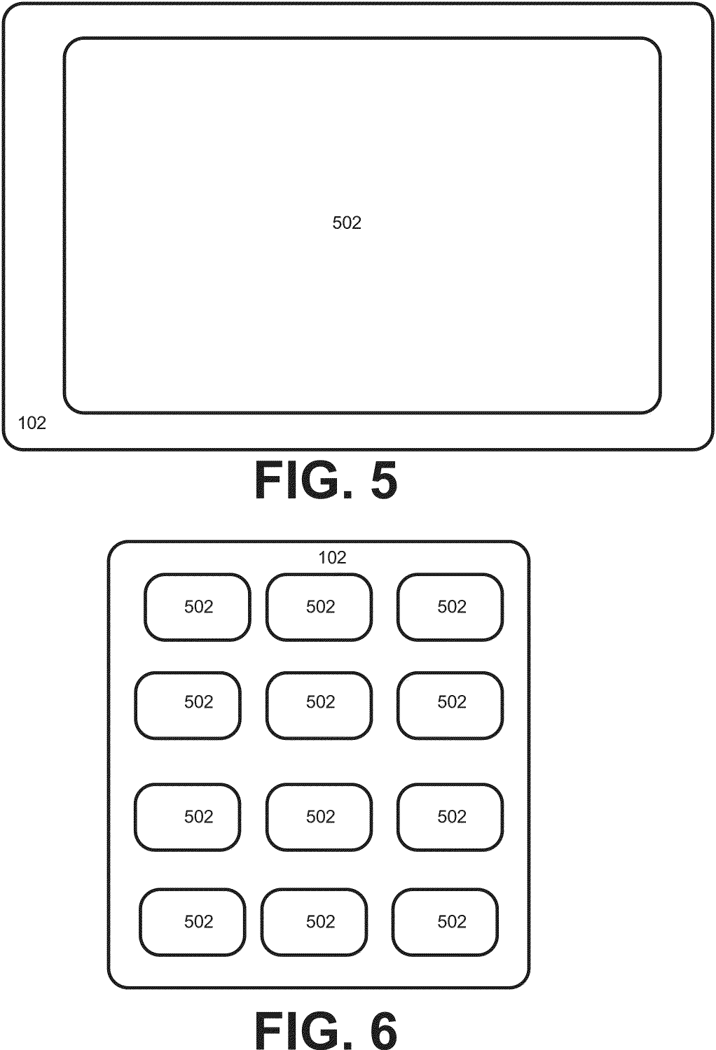

The photoceutical medical device 102 can be of different shapes and sizes so long as the photoceutical medical device is shaped and sized to deliver the photoceutical 104 to the one or more predefined target sites on the patient. As examples, the photoceutical medical device 102 may be configured to be held against (see, e.g., FIGS. 1, 3, and 4) and/or placed onto (see, e.g., FIGS. 5 and 6) one or more treatment sites (e.g., target sites) for delivery of the photoceutical 104. While the photoceutical 104 can be emitted from one or more circular emitters (as shown in FIG. 1), the emitters need not be circular. For example, the emitters can be shaped as one or more diamonds, crosses, squares, rectangles, or the like. Components of the photoceutical medical device 102 that facilitate generation and delivery of the photoceutical 104 are shown in FIG. 2. It should be understood that the photoceutical medical device 102 can include additional components to facilitate the generation and delivery of the photoceutical 104 (e.g., one or more lenses).

The photoceutical medical device 102 can include a circuit board 202 (also referred to as a printed circuit board), which can hold the light sources (which can be positioned in a manner for delivery). The circuit board 102 can interface with an emitter that facilitates the photoceutical 104 from the light source(s) and, in some instances, magnet(s) leaving the photoceutical medical device 102. Each light source can deliver a unique signal from a unique position, and these unique signals can be combined to form the photoceutical 104. The light sources can include one or more super-pulsed lasers, one or more red light emitting diodes (LEDs), and one or more infrared light emitting diodes (IRED). In some instances, the circuit board 202can include one or more magnetic sources.

The circuit board 202 can be, in some instances, a flexible circuit board, and in other instances, a rigid circuit board. The circuit board 202 can be connected to a controller 204 and a power source 206. The controller 204 can be configured to receive an input, for example an external input from a user, a memory storing instructions, and/or a processor to execute the instructions. The instructions can include programming for delivery of the photoceutical 104, including the duration of the delivery of the photoceutical 104, the start time, the stop time, the total power, based on the power delivered by each source (the types of light sources and, in some instances, the magnetic sources), and the like. Although the controller 204 can be controlled by an input, in some instances, the controller 204 can be preprogrammed such that only a start or stop button is required. In some instances, at least a portion of the controller can be external to the photoceutical medical device 102. The power source 206 can provide line power and/or battery power to power at least a portion of the photoceutical delivery device 102 (e.g., the controller 204, which can deliver power to the circuit board 202) and can include additional circuitry related to power delivery. In some instances, the photoceutical medical device 102 can include a motor (not illustrated) to incline the circuit board 202 for delivery to at least one of the predefined sites on the patient’s body. The motor can be programmed to move the circuit board to a specific incline (e.g., angled between 10 degrees and 50 degrees from vertical or horizontal relative to the orientation of the photoceutical delivery device 102) to deliver the photoceutical 104 based on the anatomical location of the muscle at the predefined target site. However, in other instances, the circuit board 202 can be inclined manually by angling the photoceutical medical device 102.

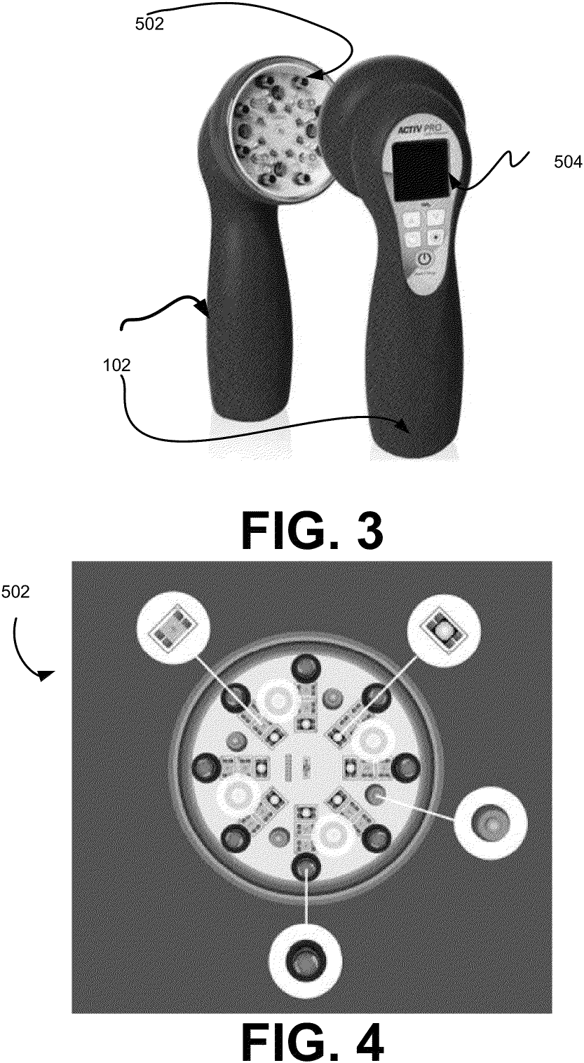

Example delivery devices are shown in FIGS. 3-6. The photoceutical delivery device 102 can have at least a portion configured to deliver the photoceutical to the muscle at each of the treatment sites (e.g., target sites). In some instances, the photoceutical delivery device 102 can have a display 504 that can facilitate configuration of the photoceutical 104 and/or starting/stopping delivery of the photoceutical 104. In FIGS. 5 and 6, different examples of flexible array devices are shown, while in FIGS. 3 and 4, an example of a probe device is shown. The examples shown in FIGS. 3-6 are just examples and not exhaustive.

### V. Methods

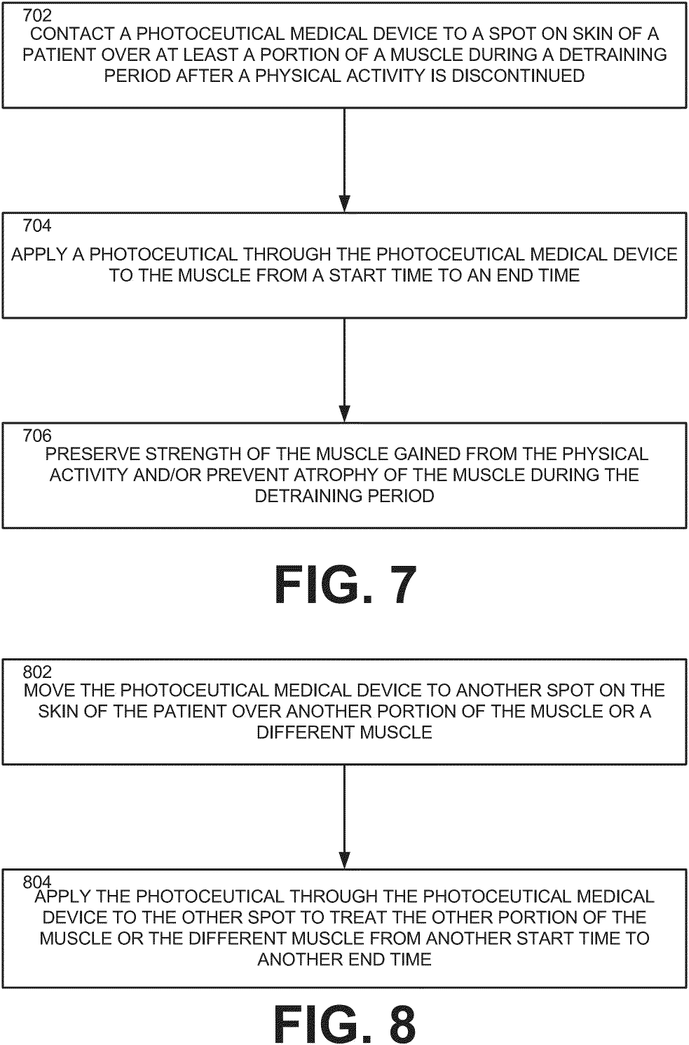

Another aspect of the present disclosure can include methods 700 and 800, as shown in FIGS. 7 and 8, for decreasing the effects caused by lack of muscle use, including loss of strength, loss of functional capacity, loss of performance, effects of muscle atrophy, preventing muscle atrophy, and/or preserving muscle morphology with a photoceutical applied through a photoceutical medical device. The methods 700 and 800 can be executed by hardware - for example, at least a portion of the system shown in FIGS. 1 and 2 and described above. The methods 700 and 800 are illustrated as process flow diagrams with flowchart illustrations. For purposes of simplicity, the methods 700 and 800 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 700 and 800. Additionally, one or more elements that implement the methods 700 and 800, such as the photoceutical delivery device 102 or the controller 204 of FIG. 2, may include a non-transitory memory and one or more processors that can facilitate the configuration and generation of the light of the photoceutical.

Shown in FIG. 7 is a method 700 for decreasing the effects caused by lack of muscle use, including loss of strength, loss of functional capacity, loss of performance, effects of muscle atrophy, preventing muscle atrophy, and/or preserving muscle morphology during a period of the lack muscle of use (e.g., a period where a patient is unable to perform a physical activity due to an injury, a disease, like COPD or ARDS, a period of hospitalization, a period of mechanical ventilation, or the like), also referred to as a detraining period. The method 700 can employ the photoceutical medical device 102 to apply the photoceutical 104 (shown in FIG. 1, for example). The photoceutical 104 can be delivered to one or more muscles identified as potentially subject to the effects caused by lack of muscle use based on specifics of the patient in a period of detraining or any other period of lack of use. For example, based on the patient, one or more leg muscles and/or one or more muscles used for respiration (e.g., chest muscles, neck muscles, the diaphragm, etc.) can be identified as targets for delivery of the photoceutical.

The photoceutical medical device 102 can be a handheld device or it can be applied to the patient’s body independently (without being handheld). The photoceutical device includes at least one super pulsed laser that can provide super-pulsed light of a first wavelength, at least two non-coherent light sources that can provide light of a second wavelength, and at least two other non-coherent light sources to provide light of a third wavelength. The light signal delivered by the photoceutical medical device can include a combination of super-pulsed light of the first wavelength, light of the second wavelength, and light of the third wavelength. For example, the first wavelength can be any wavelength between 850 nm and 950 nm, the second wavelength can be any wavelength between 800 nm and 900 nm, and the third wavelength can be any wavelength between 580 and 800 nm. The photoceutical medical device can also comprise at least two magnetic sources to provide a magnetic signal. The magnetic sources can be, for example, one or more of: a permanent magnet, a temporary magnet, and an electromagnet.

In another aspect the photoceutical medical device can include at least four super pulsed lasers, at least eight non-coherent light sources, at least eight other non-coherent light sources, and at least eight magnetic sources. The at least four super pulsed lasers can each provide the super-pulsed light of the first wavelength. The at least eight non-coherent light sources can each provide the light of the second wavelength. The at least eight other non-coherent light sources can each provide the light of the third wavelength. The at least eight magnetic sources can provide a magnetic signal (e.g., a magnetic field).

At step 702, a photoceutical medical device can be contacted to a spot on skin of a patient over at least a portion of a muscle during a detraining period after physical activity is discontinued. The muscle can be skeletal muscle, smooth muscle, or cardiac muscle. For example, based on the patient, one or more leg muscles and/or one or more muscles used for respiration (e.g., chest muscles, neck muscles, the diaphragm, etc.) can be identified as targets for application of the photoceutical.

At step 704, a dose of the photoceutical (e.g., approximately 30 J) is delivered through the photoceutical medical device to the muscle from a start time to an end time (e.g., leading to an exposure time from 30 seconds to 1 hour). The photoceutical can include at least one of a pulsed light signal, a continuous light signal, and a super-pulsed light signal. In some instances, the light signal can include a super-pulsed light signal and at least one of at least one pulsed light signal and at least one continuous light signal. In some instances, the photoceutical can also include one or more magnetic signals (making up a magnetic field). At step 706, strength of the muscle (or morphology, functional capacity, performance, and the like) gained from the physical activity is preserved and/or atrophy of the muscle is prevented during the detraining period.

FIG. 8 continues the method 700. At 802, the photoceutical medical device can be moved to another spot on the skin of the patient over another portion of the muscle or over a different muscle. This can occur after expiration of the first time period (for application of the light signal to the previous spot). The spots can be predefined according to the effects to be prevented and the function to be preserved. At 804, the photoceutical can be applied through the photoceutical medical device to the muscle or the other muscle from another start time to another end time (a second time period). The second time period can be between 30 seconds and 300 seconds. The other dose delivered for the second time period to the other site can be approximately 30 J.

### VI. Experimental

This Experiment shows that a pharmaceutical including photobiomodulation therapy (PBMT) and static magnetic field (sMF) (referred to herein as PBMT/sMF) can potentiate the effects of strength training and decrease the effects of performance loss after a 4-week detraining period. It should be understood that while leg muscles were studied herein, these results can be extended to other muscles (e.g., respiratory muscles).

### Materials and Methods

A randomized, triple-blind (volunteers, therapists, and assessors), placebo-controlled clinical trial was performed at Laboratory of Phototherapy and Innovative Technologies in Health (LaPIT) at Nove de Julho University. The study followed the ethical guidelines and was approved by the Research Ethics Committee of Nove de Julho University (protocol number 1781602), furthermore this protocol was prospectively registered at ClinicalTrials.org (NCT03858179). All volunteers signed an informed consent at the time of enrolment in this study.

### Subjects and Sample Size

The number of participants per group in the present study was calculated based on a pilot study, with 5 volunteers per group, in order to estimate the sample size. To calculate the sample size a β value of 20% and α of 5% were used.

The pilot study showed that applying PBMT/sMF during the detraining period resulted in a peak torque (primary outcome of this study) of 257.25 Nm (33.73 standard deviation) during the maximum voluntary contraction (MVC) test, whereas applying the placebo during the detraining period resulted in a peak torque of 222.05 Nm (29.87 standard deviation). The Researcher’s Toolkit (https://www.dssresearch.com/resources/calculators/sample-size-calculator-average/) was used to calculate the sample size.

Based on the aforementioned parameters used to calculate the sample, a number of 12 volunteers per group, 48 volunteers in total were used. Since the PBMT/sMF device used in the study caused no harmful thermal effects, volunteers of different skin colors were recruited. The volunteers were informed about all study procedures and asked to sign an Informed Consent Form prior to their enrollment in the study.

### Patient and Public Involvement Statement

Patients and/or the public were not involved in the design and recruitment to conduct of this study. At the end of the study, the main results were disseminated to participants by email.

### Inclusion and Exclusion Criteria

Healthy men aged from 18 to 35 years with no history of musculoskeletal injury in the hip and knee regions in the 2 months before the study, who do not regularly use pharmacological agents and/or nutritional supplements, and who complete at least 80% of the study procedures were included in the study. Volunteers who showed any musculoskeletal injury in the 2 months before the study or who become injured during the study, who regularly used any type of nutritional supplement or pharmacological agent, or who showed signs and symptoms of any neurological, metabolic, inflammatory, pulmonary, oncological, or cardiovascular disease that may limit the execution of high-intensity exercises were excluded from the study.

### Experimental Groups, Randomization and Blinding

To avoid selection bias and to ensure that all individuals were randomly allocated to any group, balanced block randomization was performed based on the primary outcome (MVC) by a researcher who had no contact with the study subjects or the other researchers involved in the project.

A researcher programmed the device (placebo or PMBT/sMF) and was instructed to not inform the volunteers or other researchers as to the type of treatment (PMBT/sMF or placebo). Therefore, the researcher responsible for the treatment was blinded to the type of treatment being administered to the volunteers. The sounds and signals emitted from the device as well as the information displayed on the screen were identical, regardless of the type of treatment (PBMT/sMF or placebo), providing the appropriate blinding of volunteers and therapists. All volunteers used opaque glasses during treatments to enhance safety and to aid in blinding. Thus, volunteers, evaluators, and therapists were blinded to maintain the triple-blind design.

Randomization labels was created through the random.org website, and a series of sealed, opaque, and numbered envelopes were used to ensure confidentiality and to determine to which experimental group each volunteer was allocated (4 different experimental groups - 12 volunteers in each group). Volunteers were allocated as described below:
PBMT/sMF+PBMT/sMF: PBMT/sMF before the strength training sessions (12 weeks, 2 times a week) and PBMT/sMF during the detraining period (4 weeks, 2 times a week).
PBMTIsMF+Placebo: PBMT/sMF before the strength training sessions (12 weeks, 2 times a week) and placebo during the detraining period (4 weeks, 2 times a week).
Placebo+PBMT/sMF: Placebo before the strength training sessions (12 weeks, 2 times a week) and PBMT/sMF during the detraining period (4 weeks, 2 times a week).
Placebo+Placebo: Placebo before the strength training sessions (12 weeks, 2 times a week) and placebo during the detraining period (4 weeks, 2 times a week).

The individuals randomly allocated to the four different groups were subjected to 12 consecutive weeks of dynamic strength training involving leg-press and knee extension exercises in leg-press and leg-extension machines, respectively, 2 times a week.

After the 12-week training period, the volunteers received the application of PBMT/sMF or placebo depending on the group to which they are allocated for 4 weeks (2 times a week) without training.

### Procedures

The protocol included 5 planned evaluation visits: baseline, 4, 8, 12, and 16 weeks. All evaluations were performed at least 24 hours before (baseline) or after 4, 8, 12, and 16 weeks after any kind of intervention (PBMT/sMF, placebo or strength training). Evaluations were performed in the morning and the exercise was performed on the same day, in the afternoon. Volunteers were evaluated and re-evaluated at the same time of day to preclude circadian effects on the findings. Volunteers were instructed to maintain their usual physical and nutritional activities, to avoid drinking alcohol, and to sleep well.

All evaluations were performed by the same researcher who was blinded to the allocation of the individuals to the different experimental groups. Evaluations were performed on days other than the strength training days.

### Maximum Voluntary Contraction (MVC) Test

The volunteers were seated in the isokinetic dynamometer chair (Biodex System 4, Biodex Medical Systems, Shirley, NY USA) at an angle of 100° between the trunk and hip, with the tested lower limb positioned at 60° knee flexion (considering 0° total knee extension) and fixed to the dynamometer seat using a belt. The other lower limb was also positioned at 100° hip flexion and fixed to the seat with a belt. The volunteers were attached to the dynamometer seat using two belts crossing their trunk. During the tests, the volunteers were instructed to place their arms across their chest, and the axis of the dynamometer was positioned parallel to the center of the knee joint.

The MVC test consisted of three, 5-second isometric contractions of the knee extensors of the lower limb. The highest value of torque assessed in the three contractions (isometric peak torque) was used for statistical analysis because this variable reflects the maximal ability of the muscle to generate force The volunteers were verbally encouraged when performing the MVC test and instructed on how to perform the test beforehand.

The MVC test was performed before starting the study (baseline), at 4, 8, and 12 weeks after starting the training period, and at 4 weeks after completing the training (the detraining period), in both lower limbs.

### One-Repetition Maximum (1RM) Test

Initially, the volunteers were instructed to briefly warm up on a stationary bicycle (INBRAMED, Brazil), at 100 rpm and without load, for 5 minutes.

The proposed range of motion was from 0° (full knee extension) to 90°, for both exercises proposed. The anatomical references for the identification of the angle of movement are the greater trochanter of the femur, lateral epicondyle of the femur, and lateral malleolus.

Before starting the test, the volunteers were familiarized with an estimated load of less than 60% 1RM. The subjective load was identified using the OMNI perceived exertion scale (0=extremely easy and 10=extremely difficult).

The one-repetition maximum was determined by a gradual increase in load until the subject was unable to perform the exercise with a full range of motion and by using the OMNI scale. The load was chosen within 5 attempts, with a 5-minute interval between attempts, to avoid metabolic disorders and interferences in test quality. Volunteers were verbally encouraged to achieve maximum effort, and the test was performed using both the leg-extension and the leg-press machines.

The 1RM test was performed before starting the study (baseline), at 4, 8, and 12 weeks after starting the training period, and at 4 weeks after completing the training (the detraining period), in both lower limbs. The training load was reset at 4 and 8 weeks after starting the training period based on 1RM test.

After 2 days of baseline evaluation, the volunteers started the strength training. The training was performed with a load of 80% 1RM, 2 times a week on non-consecutive days (72 hours apart), and consisted of 5 sets of 10 repetitions for 12 weeks (3 months), totaling 24 leg-press and leg-extension workouts. The rest interval between sets was 2 minutes and, if the participant failed to complete a set, the volunteer was instructed to continue until concentric muscle contraction failed (concentric failure). Room temperature was kept between 22° C. and 24° C., and the load was adjusted every 8 workouts (4 weeks).

### Photobiomodulation Therapy (PBMT) and Static Magnetic Fields (SMF) - PBMT/SMF

PBMT/sMF or placebo was applied before each workout and during the detraining period, depending on the group to which the volunteers was allocated. PBMT/sMF was applied bilaterally using the direct contact method with light pressure on the skin to 6 sites of the anterior thigh (2 medial, 2 lateral, and 2 central).

A 12-diode cluster, with four 905-nm laser diodes (12.5 W peak power for each diode), four 875-nm LED diodes (17.5 mW mean power for each diode), and four 640-nm LED diodes (15 mW mean power for each diode), with a static magnetic field (35 mT) manufactured by Multi Radiance MedicalⓇ (Solon, OH, USA), was used to apply the PBMT/sMF. Considering the large irradiation area used in the present project, the use of diode clusters was essential for the application of therapy.

The dose used for applications during the training and/or detraining periods was 30 Joules (J) per site (180 J per thigh). The sounds and signals emitted from the device as well as the information displayed on the screen were identical, regardless of the type of treatment (placebo or PBMT/sMF). PBMT/sMF was applied by a single researcher who was blinded to the randomization results and the volunteer group allocation. Detailed PBMT/sMF parameters are described in Table 1.

TABLE 1

Parameters for PBMT/sMF

| | |
|---|---|
| Number of lasers | 4 Super-pulsed (infrared) |
| Wavelength (nm) | 905 (±1) |
| Frequency (Hz) | -250±1 |
| Peak power (W) - each | 12.5 |
| Averae mean optical output (mW) - each | 0.3125 |
| Power density (mW/cm²) - each | 0.71 |
| Energy density (J/cm²) - each | 0.162 |
| Dose (J) - each | 0.07125 |
| Spot size of laser (cm²) - each | 0.44 |
| Number of red LEDs | 4 Red |
| Wavelength of red LEDs (nm) | 640 (±10) |
| Frequency (Hz) | 2 |
| Averae optical output mW - each | 15 |
| Power density (mW/cm²) - each | 16.66 |
| Enr density (J/cm2) - each | 3.8 |
| (J) - each. | 3.42 |
| Spot size of red LED (cm²) - each | 0.9 |
| Number of infrared LEDs | 4 Infrared |
| Wavelength of infrared LEDs (nm) | 875 (±10) |
| Frequency (Hz) | 16 |
| Average optical output (mW) - each | 17.5 |
| Power density (mW/cm²) - each | 19.44 |
| Energy density (J/cm²) - each | 4.43 |
| Dose (J) - each | 3.99 |
| Spot Size of LED (cm²) - each | 0.9 |
| Magnetic Field (mT) | 35 |
| Irradiation time per site (sec) | 228 |
| Total dose per site (J) | 30 |
| Total dose applied in muscular group (J) | 180 |
| Aperture of device (cm²) | 20 |
| Application mode | Cluster probe held stationary in skin contact with a 90-degree angle and slight pressure |

### Statistical Analysis

The “intention to treat” analysis was performed a priori. The primary outcome was MVC. The secondary outcomes were 1RM test and structural properties of the quadriceps. The normality was tested using Shapiro-Wilk test, since a normal distribution was observed, the data were expressed as mean and standard deviation (SD). In graphs, data were expressed as mean and standard error of the mean (SEM), to ensure better visualization (due to smaller error bars in the figures). The two-way repeated measures analysis of variance (ANOVA; time versus experimental group) with post-hoc Bonferroni correction, were used. Data were analyzed in both their absolute values and percentage of change compared to baseline. The significance level was set at p<0.05. The researcher that performed the statistical analysis was blinded to randomization and allocation of patients in experimental groups.

### Results

All 48 participants completed the full 16-week study. There were no drop outs. The characteristics of the volunteers are summarized in Table 2. Statistical analysis revealed that there were no significant differences (p>0.05) between the volunteers from the four experimental groups with respect to the anthropometric variables and baseline data.

TABLE 2

Anthropometric variables in absolute values

| | PBMT/sMF + PBMT/sMF | PBMT/sMF + Placebo | Placebo + PBMT/sMF | Placebo + Placebo |
|---|---|---|---|---|
| Age (years) | 25.30 ± 6.63 | 25.09 ± 5.11 | 24.42 ± 4.08 | 26.80 ± 6.91 |
| Body mass (kg) | 80.58 ± 13.52 | 81.62 ± 13.61 | 73.88 ± 14.43 | 75.21 ± 18.44 |
| Height (cm) | 173.40 ± 4.95 | 176.45 ± 7.85 | 170.92 ± 5.71 | 174.80 ± 7.32 |

Data is expressed in average and standard deviation (±).

Regarding the MVC test, the PBMT/sMF+PBMT/sMF group showed an increase statistically significant when compared to the Placebo+Placebo group at the 4^{th}, 8^{th}, 12^{th}, and 16^{th} week. It is important to highlight that the group treated with PBMT/sMF only in the detraining (Placebo+PBMT/sMF group) showed an increase statistically significant in MVC when compared to the Placebo+Placebo group, at the 16^{th} week. In the 1RM tests, the group that received PBMT/sMF throughout the whole study (PBMT/sMF+PBMT/sMF) showed a statistically significant increase in leg press and leg extension exercises compared to Placebo+Placebo group in both the training and detraining periods.

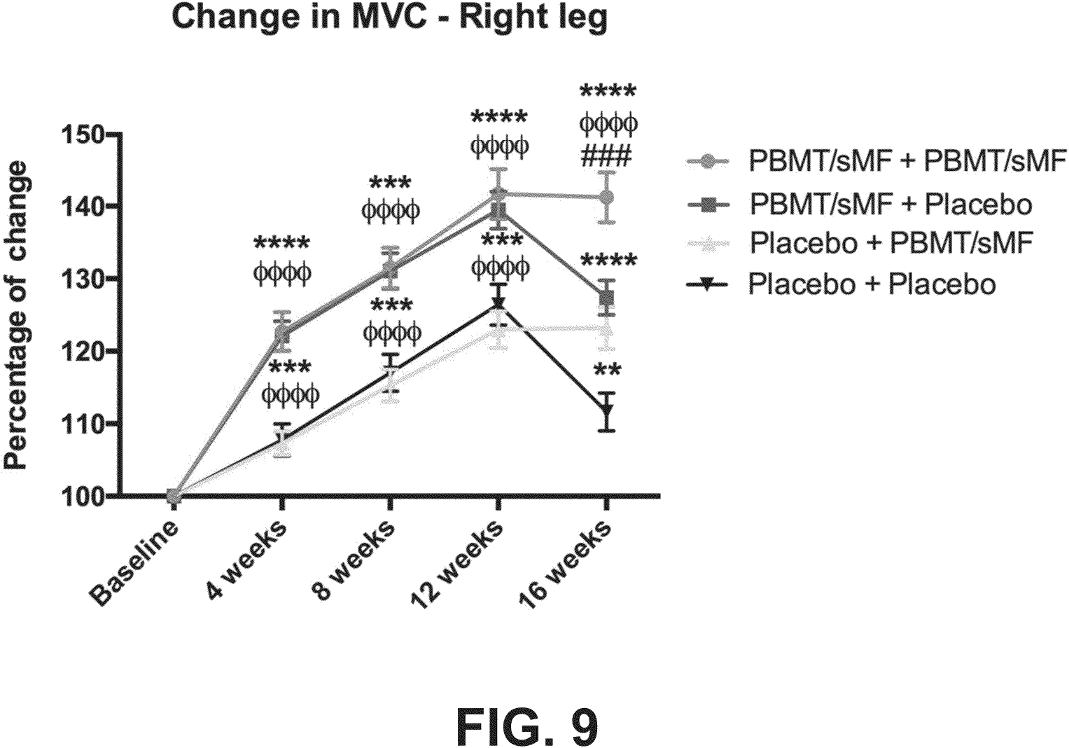

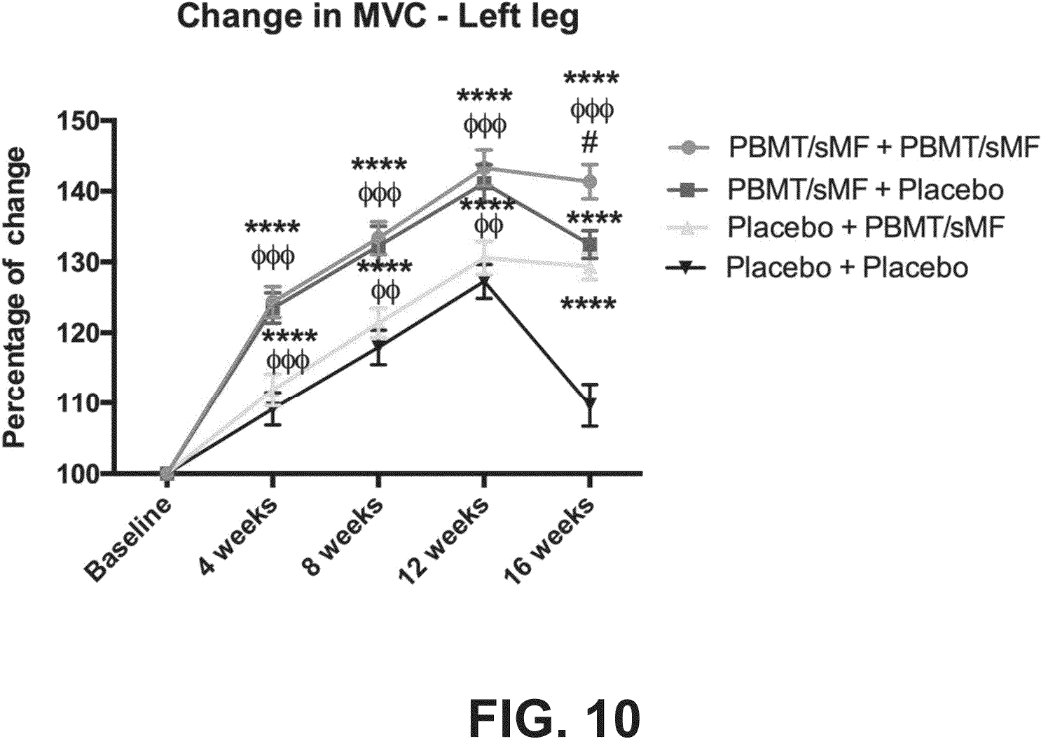

FIG. 9 (right leg) and FIG. 10 (left leg) show the percentage of change in MVC test at all evaluated time-points. During the training, the PBMT/sMF+PBMT/sMF group showed a statistically significant difference of 15% at 4^{th}, 8^{th} and 12^{th} week, compared to the Placebo+Placebo group. In the detraining period (16^{th} week), the group that received PBMT/sMF only in the training phase (PBMT/sMF+Placebo) showed a percentage of change statistically significant compared to the Placebo+Placebo group, with a difference of around 20%. However, when the PBMT/sMF was applied throughout the whole study (PBMT/sMF+PBMT/sMF) this difference was greater, around 30% compared to the Placebo+Placebo group at the 16^{th} week. It is important to note that, the group treated with PBMT/sMF only in the detraining (Placebo+PBMT/sMF group) also showed a percentage of change statistically significant compared to the Placebo+Placebo group, at the 16^{th}week.

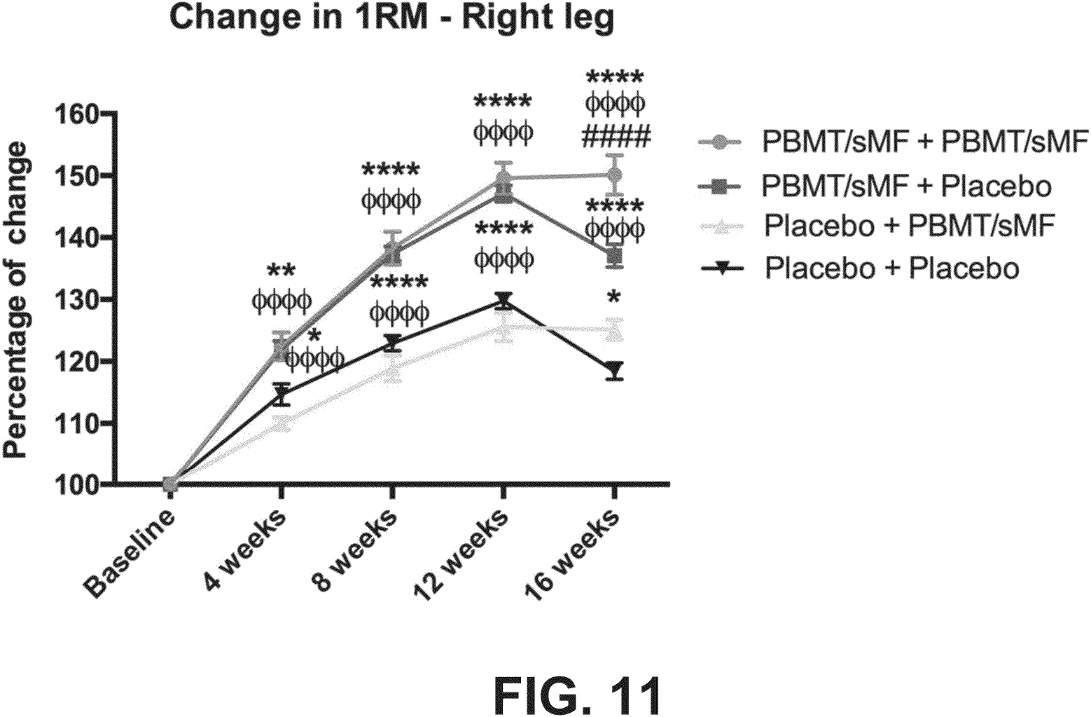

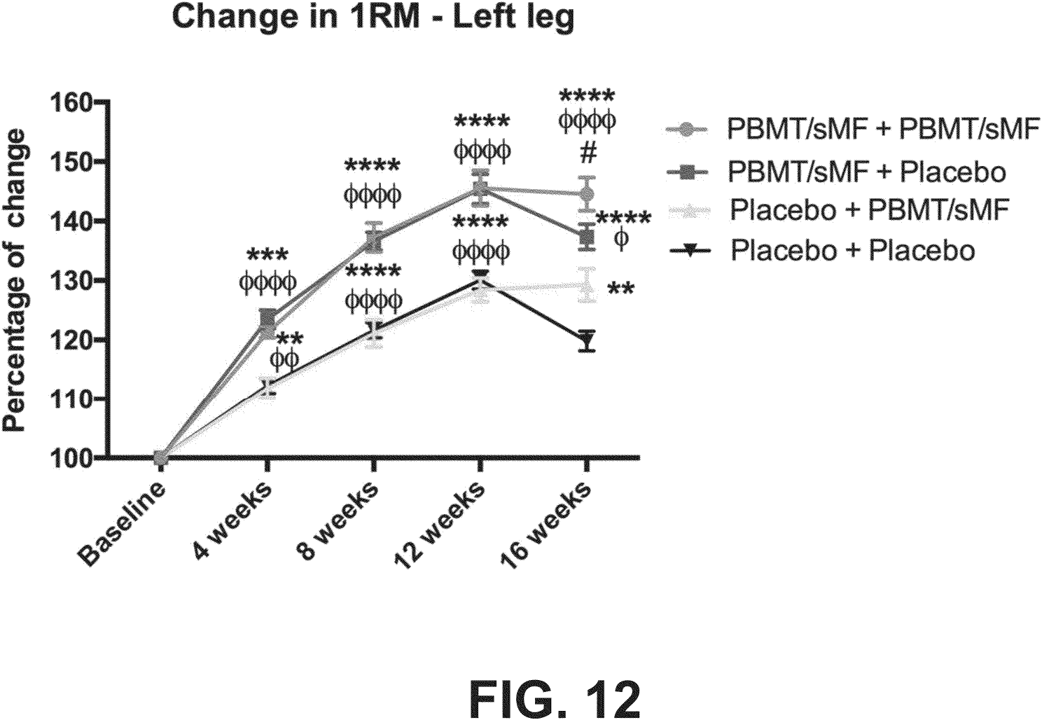

FIG. 11 (right leg) and FIG. 12 (left leg) graphically represent the percentage of change in 1RM test on leg extension in the study participants. The PBMT/sMF+PBMT/sMF group showed a percentage of change statistically significant compared to the Placebo+Placebo group at all experimental time-points, with a difference of 20% at the end of the training phase and 30% after 4 weeks without training (detraining period). Moreover, PBMT/sMF+PBMT/sMF group also had an increase in percentage of change compared to the PBMT/sMF+Placebo group, at the 16^{th} week. It is noteworthy that, the group treated with PBMT/sMF only in the detraining (Placebo+PBMT/sMF group) showed a percentage of change statistically significant in relation to the Placebo+Placebo group, around 8% at the 16^{th} week.

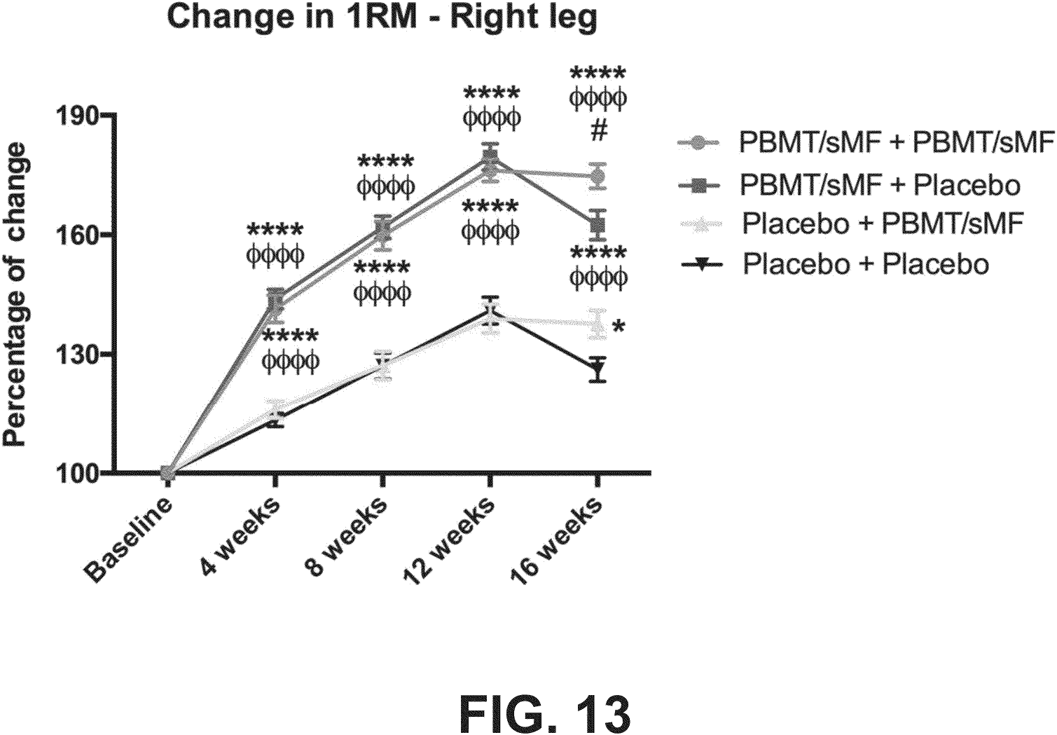

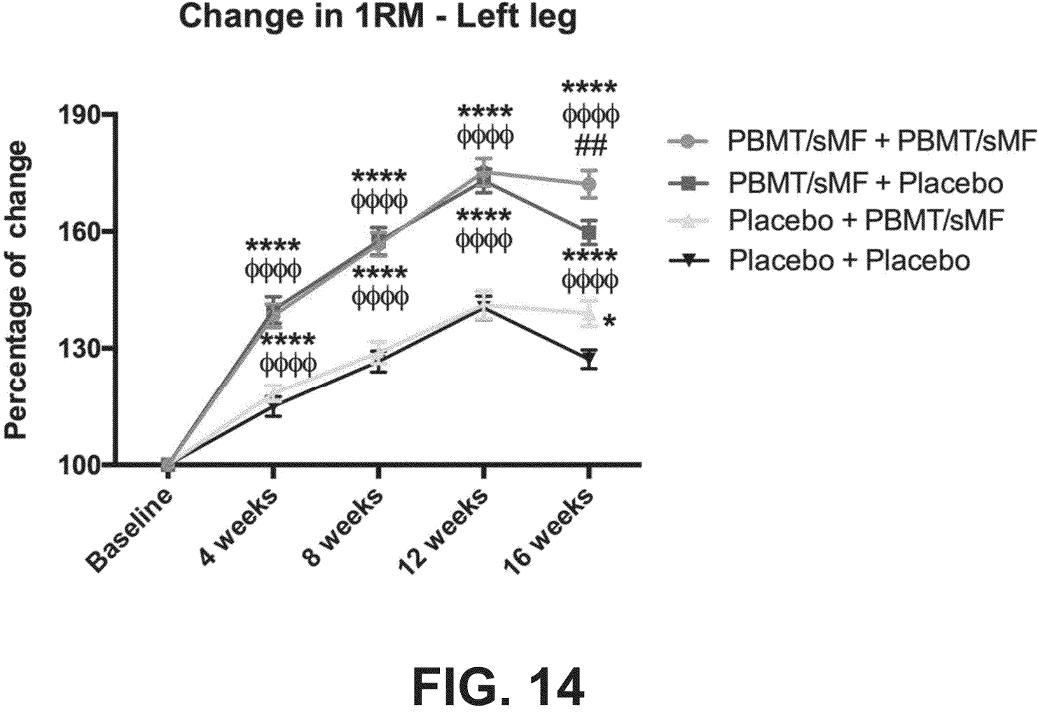

FIG. 13 (right leg) and FIG. 14 (left leg) show the percentage of change in 1RM test in the leg press. The group treated with PBMT/sMF throughout the whole study (PBMT/sMF+PBMT/sMF group) demonstrated a percentage of change statistically superior (35% difference at the end of the training phase and around 47% after 4 weeks without training) compared to the Placebo+Placebo group. Compared to the PBMT/sMF+Placebo group, the PBMT/sMF+PBMT/sMF group also showed a percentage of change statistically significant, around 12% at the 16^{th} week. The PBMT/sMF applied only in the detraining (Placebo+PBMT/sMF group) resulted in a percentage of change statistically significant, around 11% compared to the Placebo+Placebo group at the 16^{th} week.

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

The following is claimed:

1. A method comprising:
contacting a photoceutical medical device to a spot on skin of a patient over at least a portion a muscle during a detraining period after a physical activity is discontinued; and
applying a photoceutical through the photoceutical medical device to the muscle from a start time to an end time to preserve strength of the muscle gained from the physical activity and/or prevent atrophy of the muscle during the detraining period,
wherein the photoceutical comprises at least one of a pulsed light signal, a continuous light signal, and a super-pulsed light signal.

2. The method of claim 1, wherein the photoceutical comprises a super-pulsed light signal and at least one of a pulsed light signal and a continuous light signal.

3. The method of claim 1, wherein the photoceutical further comprises a magnetic field.

4. The method of claim 1, wherein the start time and end time least to an exposure time from 30 seconds to 1 hour.

5. The method of claim 4, further comprising:
moving the photoceutical medical device to another spot on the skin of the patient over at least another portion of the muscle or at least a portion of another muscle; and
applying the photoceutical through the photoceutical medical device to the other portion of the muscle or the portion of the other muscle from another start time to another end time.

6. The method of claim 1, wherein the photoceutical further comprises a static magnetic field from 5 mT to 1 T.

7. The method of claim 1, wherein the photoceutical medical device comprises:
at least one super pulsed laser to provide the super-pulsed light signal; and
at least two non-coherent light sources to provide the pulsed light signal and/or the continuous light signal.

8. The method of claim 7, wherein:
the at least one super pulsed laser provides the super-pulsed light signal at a first wavelength;
the at least two non-coherent light sources provide the pulsed light signal and/or the continuous light signal at a second wavelength and a third wavelength.

9. The method of claim 1, wherein the photoceutical comprises super-pulsed light of a first wavelength from at least one super-pulsed light source, pulsed and/or continuous light of a second wavelength from at least two non-coherent light sources, and pulsed and/or continuous light of the third wavelength from at least two other non-coherent light sources.

10. The method of claim 9, wherein the first wavelength is between 850 nm and 950 nm, the second wavelength is between 800 nm and 900 nm, and the third wavelength is between 580 nm and 800 nm.

11. The method of claim 9, wherein the photoceutical medical device comprises:
at least four super-pulsed lasers, each configured to provide the super-pulsed light of the first wavelength;
at least eight pulsed and/or continuous light sources, each configured to provide the light of the second wavelength;
at least eight other pulsed and/or continuous light sources, each configured to provide the light of the third wavelength; and
at least eight magnetic sources to provide a magnetic signal.

12. The method of claim 1, wherein the muscle is any soft tissue within the patient’s body that contracts to change a length and/or a shape of the soft tissue.

13. The method of claim 12, wherein the muscle is skeletal muscle.

14. The method of claim 1, further comprising:
selecting the spot on skin of a patient over at least a portion a muscle; and
selecting another spot on the skin of the patient over at least a portion of another muscle or at least another portion of the muscle.

15. A photoceutical medical device comprising:
a circuit board comprising:
a plurality of light sources to provide a light signal comprising:
at least one super pulsed laser to provide super-pulsed light of a first wavelength,
at least two non-coherent light sources to provide pulsed and/or continuous light of a second wavelength,
at least two other non-coherent light sources to provide pulsed and/or continuous light of a third wavelength,
wherein the light signal comprises at least one of the super-pulsed light of the first wavelength, the pulsed and/or continuous light of the second wavelength, and the pulsed and/or continuous light of the third wavelength; and
at least two magnets to provide a magnetic signal,
wherein the light signal and the magnetic signal are delivered as a photoceutical to a spot on skin of a patient over at least a portion a muscle during a detraining period after a physical activity is discontinued from a start time to an end time to preserve strength of the muscle gained from the physical activity and/or prevent atrophy of the muscle during the detraining period; and
a power source.

16. The photoceutical medical device of claim 15, wherein the light signal comprises the super-pulsed light of the first wavelength, the pulsed and/or continuous light of the second wavelength, and the pulsed and/or continuous light of the third wavelength.

17. The photoceutical medical device of claim 15, wherein the light source device is a probe device or a flexible array device.

18. The photoceutical medical device of claim 15, wherein the circuit board interfaces with an emitter that facilitates the photoceutical leaving the photoceutical medical device.

19. The photoceutical medical device of claim 15, wherein the plurality of light sources further comprises:
at least four super pulsed lasers, each configured to provide the super-pulsed light of the first wavelength,
at least eight light sources, each configured to provide the light of the second wavelength, and
at least eight other light sources, each configured to provide the light of the third wavelength.

20. The photoceutical medical device of claim 19, wherein the at least two magnets comprise at least eight magnets to provide the magnetic signal.
